(19) United States
(12) Patent Application Publication
(10) Pub. No.: US 2024/0206484 A1
(43) Pub. Date: Jun. 27, 2024

(54) METHOD OF DEVELOPMENT OF SELECTIVE PROBIOTIC GROWTH PROMOTORS AND SELECTIVE PATHOGEN GROWTH INHIBITORS FROM KIMCHI FERMENTATION

(71) Applicant: Hannip Inc., Cheongju-si (KR)

(72) Inventors: HeeChan CHOI, Cheongju-si (KR); Darrick Soo-Hong-Luke KIM, Wayne, IL (US)

(73) Assignee: Hannip Inc., Cheongju-si (KR)

(21) Appl. No.: 18/086,954

(22) Filed: Dec. 22, 2022

(51) Int. Cl. A23B 4/22; A23B 7/10; A23L 33/135

(52) U.S. Cl. CPC A23B 4/22; A23B 7/105; A23L 33/135; C12N 1/205

(57) ABSTRACT

A concentrated fermented kimchi solution is provided. The concentrated fermented kimchi solution (BKE-X1 and BKE-X2) has the benefit of inhibiting growth of pathogenic gut bacteria while promoting the growth of probiotics beneficial to the human body, and is therefore expected to help form a healthy gut microbiome environment to avert or prevent the occurrence of illnesses caused by pathogenic bacteria.

### TECHNICAL FIELD

The present invention relates to a concentrated kimchi solution fermented using a ceramic comprising germanium. More specifically, the present invention relates to a concentrated fermented kimchi solution which exhibits benefits of promoting growth of beneficial gut bacteria (probiotics) and inhibiting growth of (pathogenic) gut bacteria, and a method for preparing the same.

### BACKGROUND ART

Fermentation is a method for preserving foods prone to spoilage, and has been used for more than a thousand years across numerous nations and regions. Fermented foods are recognized for nutrition and unique flavors, and various fermented foods are consumed on a daily basis throughout the world. Notable fermented foods include cheese, yoghurt, kefir, wine, beer, dwenjang, sauerkraut, kombucha, pozole, and kimchi.

Kimchi is a traditional Korean fermented food, and is recognized the world over for its health benefits owing to high probiotics content. Kimchi may be made from various ingredients, including Baechu cabbage, cucumbers, dried radish greens, spring onions, and the like. Among the most popular kinds of kimchi in Korea is “Baechu cabbage kimchi” made from Korean Baechu cabbage. Baechu cabbage kimchi is highly consumed and easily cooked, and therefore its fermentation processes have been studied numerous times.

Fermentation of kimchi involves various bacterial species, including Leuconostoc spp., Lactococcus spp., Lactobacillus spp., and Weissella spp. Of these, the lactic acid bacteria (LAB) species which both ferment kimchi and produce lactic acid are expected to have probiotic characteristics and health benefits.

L. mesenteroides is dominant in early fermentation of Baechu cabbage kimchi, with small amounts of Lactobacillus spp. and Weissella spp. as well. As fermentation progresses, Lactobacillus spp. (more specifically, L. plantarum) becomes dominant, while L. mesenteroides and Weissella spp. decrease. Over extended periods (several months) or fermentation, Weissella spp. reappears, supplanting L. plantarum and becoming the sole surviving dominant species.

Lactic acid has been suggested as being a core factor in the fermentation of Baechu cabbage kimchi, regulating growth patterns of these organisms through pH changes and determining the dominant species growth pattern transition from L. mesenteroides to L. plantarum. L. mesenteroides is reported as the dominant species at relatively high pH (5.0 to 6.0) in the early stages of Baechu cabbage kimchi fermentation. However, it has been proposed that due to the lactic acid produced by L. plantarum, the growth environment becomes acidic, resulting in a transition of dominant species from L. mesenteroides to L. plantarum, ultimately inhibiting the survival of L. mesenteroides. Further, it has been reported that Weissella spp. becomes the dominant species as the growth environment becomes acidic due to the maturing of Baechu cabbage kimchi and release of lactic acid by L. plantarum.

Meanwhile, the human intestinal mucosa is comprised of the Lamia propria, which are epithelial cells, and the lamina muscularis mucosae, and is home to upwards of 1,000 types of microbe. The number of microbes making up the human gut microbiome is estimated to be at least 10 times the number of cells making up the human body, most of these being unknown anaerobic strains. Various pathogenic bacteria infiltrate and live inside the human gut, causing disease, and it has been reported that infiltration of pathogenic bacteria into the gut and their growth gives rise to imbalances in the gut microbiome, which can cause substantial problems to the health of the human body.

Pathogenic bacteria frequently infiltrate the gastrointestinal system and have substantially harmful effects on the human body. Representative types of pathogenic bacterium include Escherichia coli, Salmonella, Klebsiella, Citrobacter spp., Clostridium spp., Staphylococcus aureus, Proteus mirabilis and Enterobacter spp. Some of these can cause diarrhea, fever, gastrospasm, ulcerative colitis, Crohn's disease, obesity, diabetes, colorectal cancer, and rheumatoid arthritis.

### SUMMARY OF THE INVENTION

### Technical Problem

Through efforts to develop a method for creating a beneficial gut microbiome, the present inventors have verified that a concentrated kimchi solution fermented using a ceramic comprising germanium exhibits benefits of promoting growth of beneficial gut bacteria (probiotics) and inhibiting growth of (pathogenic) gut bacteria, thereby completing the present invention.

Accordingly, an object of the present invention is to provide a method for preparing a concentrated fermented kimchi solution.

Another object of the present invention is to provide a concentrated fermented kimchi solution prepared using the above method.

### Technical Solution

Through efforts to develop a method for creating a beneficial gut microbiome, the present inventors have verified that a concentrated kimchi solution fermented using a ceramic comprising germanium exhibits benefits of promoting growth of beneficial gut bacteria (probiotics) and inhibiting growth of (pathogenic) gut bacteria.

The present invention relates to a method for preparing a concentrated fermented kimchi solution, and a concentrated fermented kimchi solution prepared using the method.

In the following, the present invention will be described in further detail.

One aspect of the present invention relates to a method for preparing a concentrated fermented kimchi solution, the method comprising the following steps: a step of placing kimchi to which a kimchi seasoning paste has been applied into a container with germanium-containing ceramic pieces, then adding salt water and fermenting for 20 to 30 hours at room temperature; and a step of mashing the kimchi at day 3 or 15 of fermentation, neutralizing, and vacuum concentrating at 80 to 90° C.

The method may further comprise the following steps: a step of cutting and brining the main ingredient; a step of preparing the kimchi seasoning paste; and a step of applying the kimchi seasoning paste onto the brined main ingredient.

In the following, each step will be described in further detail.

### [Step of Cutting and Brining the Main Ingredient]

Fresh main ingredients for kimchi are sorted and rinsed, then prepared by cutting to size according to the type of kimchi being made. Preferably, in one example Baechu cabbage is submerged in a brine (containing, for example, sun-dried salt, glasswort salt, or pink Himalayan salt, etc.) for 5 to 10 hours, carrying out the brining process with care so as not to excessively brine the Baechu cabbage.

The main ingredient may be Baechu cabbage, white radish, summer radish, spring onion, leaf mustard, and the like, but is not limited hereto.

The kimchi may be a fermented product of green leafy vegetables, such as Baechu cabbage kimchi, water kimchi, white radish kimchi, summer radish kimchi, ponytail radish kimchi, cucumber kimchi, spring onion kimchi, leaf mustard kimchi, and the like. Preferably, the kimchi is a Baechu cabbage kimchi made with Baechu cabbage, but is not limited hereto.

In the present invention, “kimchi” is an inclusive term referring to a fermented product based on various leafy green vegetables, including varieties of Baechu cabbage, white radish, leaf mustard, onion (bulb, spring onion, leek, scallion), and cucumber.

### [Step of Preparing the Kimchi Seasoning Paste]

The kimchi seasoning paste is characterized in that it is prepared by mixing shredded white radish, spring onion, celery, water parsley, fermented shrimp, red pepper flakes, red pepper power paste, salted fermented yellow corvina, salt, corn syrup, mashed ginger, mashed garlic, and mixing paste (Indian rice:Brown rice:Glutinous rice powder=1:1:1, stirred into 100 g warm water).

Ingredients may be added or removed from the kimchi seasoning paste depending on the type of kimchi.

### [Step of Applying the Kimchi Seasoning Paste to the Brined Main Ingredient]

The kimchi seasoning paste prepared as described above is applied evenly over the brined main ingredient to complete the kimchi.

[Step of Placing the Kimchi with Seasoning Paste Applied into a Container with Germanium-Containing Ceramic Pieces, Adding Salt Water, and Fermenting to 20 to 30 Hours at Room Temperature]

The kimchi with seasoning paste applied is placed in a container with germanium-containing ceramic pieces, followed by addition of salt water and fermentation for 24 hours at room temperature (1 to 35° C.).

The germanium-containing ceramic pieces may be prepared using various methods known to the art, for example, by mixing germanium powder with white clay and baking at 700 to 900° C., then baking a second time at 1,200 to 1,300° C., but the method is not limited hereto.

Further, there is no limit to the shape, size, surface area and thickness to the germanium-containing ceramic pieces, and suitable values may be decided by a person skilled in the art using ordinary and commonly used optimization techniques, sound professional judgment, and other factors evident to a person skilled in the art.

In the present invention, “germanium” is a naturally occurring trace element, which may be applied to various fields from electronics to nutritional supplements. Organic germanium has been reported to be a therapeutic agent having anti-cancer, anti-tumor, anti-aging, anti-viral, and anti-inflammatory effects. Further, organic germanium has exhibited efficacy in cancer and arthritis treatment, and has exhibited efficacy in improving immune function in pathological patients in pre-clinical trials.

The germanium may be included at a concentration of 0.01% to 99%.

### [Step of Mashing the Baechu Cabbage at Day 3 Through 15 of Fermentation, Neutralizing, and Vacuum Concentrating at 80 to 90° C.]

After fermentation for 24 hours at room temperature, the kimchi is fermented at low temperature (3 to 5° C.). Here, the cabbage is mashed on day 3 or 15 of fermentation, rinsed with water, and neutralized to a pH of 7.0. This step is characterized in that the mashed and neutralized kimchi is filtered (2×), then vacuum concentrated (10×) at 80 to 85° C.

In the present invention, the concentrated fermented kimchi solution prepared through the above-described steps on day 3 of fermentation was assigned the name “BKE-X1.”

According to one example of the present invention, the BKE-X1 of the present invention promotes explosive growth of L. mesenteroides, L. plantarum and Weisella, and this effect is impacted negligibly by changes in pH.

Further, in the present invention, the concentrated fermented kimchi solution prepared through the above-described steps on day 15 of fermentation was assigned the name “BKE-X2.”

According to another example of the present invention, the BKE-X2 of the present invention selectively substantially inhibits growth of L. mesenteroides and Weisella, but promotes growth of L. plantarum and L. brevis, and this effect is not impacted by changes in pH.

These results imply that L. mesenteroides produces factors promoting growth of L. plantarum, L. brevis, W. koreensis, W. ciberia, and the like, while L. plantarum produces selective growth-inhibiting factors for L. mesenteroides and Weisella. Further, it is implied that pH is a not a key contributing factor for determining the transition in growth pattern from L. mesenteroides-dominant to L. plantarum-dominant.

In summary, the fermentation process of kimchi can be defined as follows:

Growth of L. mesenteroides, Lactobacillus spp. and Weisella spp. occurs in early fermentation of kimchi. As the kimchi ferments, selective growth promoters for Lactobacillus spp. appear, and simultaneously, selective growth inhibitors for L. mesenteroides and Weisella spp. are generated. As fermentation progresses, L. plantarum becomes the dominant species, and in cases where there is an abundance of L. plantarum, L. brevis may also become a dominant species. As the kimchi matures, the selective growth inhibiting activity is slightly decreased and/or decomposed, leading to a disappearance of L. plantarum and Weisella spp. again becoming the dominant organism.

Another aspect of the present invention relates to a concentrated fermented kimchi solution prepared using the method described above.

In one example of the present invention, the concentrated fermented kimchi solution may be prepared through a vacuum concentration step on day 3 of fermentation (BKE-X1).

The concentrated fermented kimchi solution (BKE-X1) contains probiotics and growth promotion factors for other bacteria (psychrotrophic bacteria, pathogenic bacteria).

In the present invention, “growth promotion” relates to a function for regulating or enhancing the growth of beneficial bacteria found inside or outside the human body. The term ‘probiotics’ includes beneficial bacteria found inside the gut, which make up the human or gut microbiome.

In the present invention, “probiotics” is a general concept relating to live bacteria which exhibit beneficial health effects, and these are also referred to as lactobacilli. Probiotics are a general term referring to gut bacteria which are beneficial to health, and probiotics are being used in various cosmetic and health functional food applications.

Specifically, the probiotics of the present invention may be, but are not limited to, Leuconostoc mesenteroides, Lactobacillus spp., Weisella spp., Limosilactobacillus fermentum, Bacillus clausii, Bacillus coagulans, Bifidobacterium animalis, Bifidobacterium bifidum, Bifidobacterium breve, Bifidobacterium longum, Clostridium butyricum, Escherichia coli Nissle 1917, Lacticaseibacillus casei, Lacticaseibacillus paracasei, Lacticaseibacillus rhamnosus, Lactiplantibacillus plantarum, Lactobacillu acidophilus, Lactobacillus crispatus, Lactobacillus delbrueckii subsp. bulgaricus, Limosilactobacillus reuteri, Propionibacterium freudenreichii, Saccharomyces boulardii, Saccharomyces cerevisiae, Streptococcus thermophilus, and the like.

In one example of the present invention, the probiotics exhibiting a growth promotion effect due to BKE-X1 may be L. mesenteroides, L. plantarum, L. brevis, L. koreensis and W. ciberia.

In another example of the present invention, the concentrated fermented kimchi solution may be prepared through a vacuum concentration step on day 15 of fermentation (BKE-X2).

The concentrated fermented kimchi solution (BKE-X2) has a probiotics (lactobacillus) growth-promoting effect.

Further, the concentrated fermented kimchi solution (BKE-X2), unlike BKE-X1, has an effect of inhibiting growth of harmful gat bacteria.

In the present invention, “growth inhibition” refers to a function by which growth of harmful bacteria found inside or outside the human body can be regulated or inhibited. The term “gut bacteria” includes all bacteria which infiltrate the gut microbiome and are found inside the human body or gut.

In the present invention, gut bacteria (microbes) may include, but are not limited to, Leuconostoc mesenteroides, Weisella spp., Achromobacter spp., Acidaminococcus fermentans, Acinetobacter calcoaceticus, Actinomyces spp., Actinomyces viscosus, Actinomyces naeslundii, Aeromonas spp., Aggregatibacter actinomycetemcomitans, Anaerobiospirillum spp., Alcaligenes faecalis, Arachnia propionica, Bacillus spp., Bacteroides spp., Bacteroides gingivalis, Bacteroides fragilis, Bacteroides intermedius, Bacteroides melaninogenicus, Bacteroides pneumosintes, Bacterionema matruchotii, Bifidobacterium spp., Buchnera aphidicola, Butyrivibrio fibrosolvens, Campylobacter spp., Campylobacter coli, Campylobacter sputorum, Campylobacter upsaliensis, Candida albicans, Capnocytophaga spp., Clostridium spp., Citrobacter freundii, Clostridium difficile, Clostridium sordellii, Corynebacterium spp., Cutibacterium acnes, Eikenella corrodens, Enterobacter cloacae, Enterococcus spp., Enterococcus faecalis, Enterococcus faecium, Escherichia coli, Eubacterium spp., Faecalibacterium spp., Flavobacterium spp., Fusobacterium spp., Fusobacterium nucleatum, Gordonia spp., Haemophilus parainfluenzae, Haemophilus paraphrophilus, Lactobacillus spp., Leptotrichia buccalis, Methanobrevibacter smithii, Morganella morganii, Mycobacteria spp., Mycoplasma spp., Micrococcus spp., Mycobacterium chelonae, Neisseria spp., Neisseria sicca, Peptococcus spp., Peptostreptococcus spp., Plesiomonas shigelloides, Porphyromonas gingivalis, Propionibacterium spp., Providencia spp., Pseudomonas aeruginosa, Roseburia spp., Rothia dentocariosa, Ruminococcus spp., Ruminococcus bromii, Sarcina spp., Staphylococcus aureus, Staphylococcus epidermidis, Streptococcus anginosus, Streptococcus mutans, Streptococcus oralis, Streptococcus pneumoniae, Streptococcus sobrinus, Streptococcus viridans, Torulopsis glabrata, Treponema denticola, Treponema refringens, Veillonella spp., Vibrio spp., Vibrio sputorum, Wolinella succinogenes, Yersinia enterocolitica, and the like.

In the present invention, pathogenic gut bacteria may include, but are not limited to, Escherichia coli, Proteus mirabilis, Staphylococcus aureus, Bacillus cereus, and the like.

In one example of the present invention, the probiotics exhibiting a growth promotion effect due to BKE-X2 may be L. plantarum, L. brevis, L. rhamnosus, L. acidophilus and L. fermentum.

In one example of the present invention, the gut bacteria exhibiting a growth inhibition effect due to BKE-X2 may be L. mesenteroides, W. koreensis, W. ciberia, E. coli, P. mirabilis, S. aereus and B. cereus.

Redundant description as to the concentrated fermented kimchi solution and method for preparing the same will be omitted with consideration for the complexity of the present specification.

### BENEFIT(S) OF THE INVENTION

The present invention relates a concentrated fermented kimchi solution. The concentrated fermented kimchi solution (BKE-X2) according to the present invention has the benefit of inhibiting growth of pathogenic gut bacteria while promoting the growth of probiotics beneficial to the human body, and is therefore expected to help form a healthy gut microbiome environment to avert or prevent the occurrence of illnesses caused by pathogenic bacteria.

### BRIEF DESCRIPTION OF THE DRAWINGS

FIG. 1 shows germanium-containing ceramic blocks of varying size and shape, produced in accordance with one example of the present invention.

FIGS. 2a, 2b, 2c, 2d, and 2e show results of tests to determine the effect of the BKE-X1 according to one example of the present invention on the growth of the kimchi bacteria L. mesenteroides (FIG. 2a), L. plantarum (FIG. 2b), L. brevis (FIG. 2c), W. cibaria (FIG. 2d), and W. koreensis (FIG. 2e) (Control: culture medium without BKE-X1; 10×, 20× or 30×: concentration of BKE-X1; error bar indicates results that are statistically significantly different at p<0.05; all tests repeated at least 3 times).

FIGS. 3a, 3b, 3c, 3d, 3e, 3f, 3g, and 3h show results of tests to determine the effect of the BKE-X2 according to one example of the present invention on the growth of the kimchi bacteria L. mesenteroides (FIG. 3a), W. cibaria (FIG. 3b), W. koreensis (FIG. 3c); L. plantarum (FIG. 3d) and L. brevis (FIG. 3e) which are both kimchi bacteria and gut bacteria; and the gut bacteria L. rhamnosus (FIG. 3f), L. acidophilus (FIG. 3g) and L. fermentum (FIG. 3h). (Control: culture medium without BKE-X2; 10×, 20× or 30×: concentration of BKE-X2; error bar indicates results that are statistically significantly different at p<0.05; all tests repeated at least 3 times).

FIGS. 4a and 4b show results of tests to determine the effect of lactic acid treated with the BKE-X1 or BKE-X2 according to one example of the present invention on the growth of the kimchi bacteria L. mesenteroides (FIG. 4a) and L. plantarum (FIG. 4b). (Control: culture medium without BKE-X1 or BKE-X2; LA: lactic acid; BKE-X1 and BKE-X2 used at a concentration of 3×; error bar indicates results that are statistically significantly different at p<0.05; all tests repeated at least 3 times).

FIGS. 5a, 5b, and 5c show results of tests to determine the effect of the BKE-X2 according to one example of the present invention on the growth of the pathogenic bacteria E. coli (FIG. 5a), P. mirabilis (FIG. 5b), and S. aureus (FIG. 5c) (Control: culture medium without BKE-X2; 10× or 20×: concentration of BKE-X2; error bar indicates results that are statistically significantly different at p<0.05; all tests repeated at least 3 times).

FIGS. 6a, 6b, 6c, and 6d shows results of growth inhibition ring tests for the pathogenic bacteria E. coli (FIG. 6a), P. mirabilis (FIG. 6b), S. aureus (FIG. 6c), and B. cereus (FIG. 6d) grown in agar according to one example of the present invention (10× concentration shown on left, 30× on right; inhibition zone measured in mm)

FIG. 7a shows results of Kirby-Bauer test results for the BKE-X1 according to one example of the present invention with respect to the bacteria L. mesenteroides, L. plantarum, L. brevis, W. cibaria, and W. koreensis which are associated with kimchi fermentation (from left to right: control, and BKE-X1 at concentrations of 10×, 20× and 30×)

FIG. 7b shows results of Kirby-Bauer test results for the BKE-X2 according to one example of the present invention with respect to the bacteria L. mesenteroides, L. plantarum, L. brevis, W. cibaria, and W. koreensis which are associated with kimchi fermentation (from left to right: control, and BKE-X2 at concentrations of 10×, 20× and 30×)

FIG. 7c shows results of Kirby-Bauer test results for the BKE-X2 according to one example of the present invention with respect to the gut bacteria L. plantarum, L. brevis, L. rhamnosus, L. acidophilus, and L. fermentum (L. plantarum and L. brevis: control, and 10×, 20× and 30× concentrations of BKE-X2 from left to right; L. rhamnosus, L. acidophilus and L. fermentum: control, and 10× and 20× concentrations of BKE-X2 from left to right)

### DETAILED DESCRIPTION

In the following, the present invention will be described in further detail through examples. These examples are intended solely to describe the present invention in further detail, and it shall be self-evident to a person skilled in the art that the scope of the present invention is not limited by these examples.

### Preparation Examples

### Preparation Example 1. Ingredients and Methods

Leuconostoc mesenteroides (L. mesenteroides, KCKM 0598), Lactobacillus plantarum (L.

Plantarum, KCKM 0596), Lactobacillus brevis (L. brevis, KCKM 0699), Weissella koreensis (W. koreensis, KCKM 0605) and Weissella cibaria (W. cibaria, KCKM 1130) were purchased from the World Institute of Kimchi, while Escherichia coli (E. coli, KRIBB 2441), Proteus mirabilis (P. mirabilis, KRIBB 2510), and Bacillus cereus (B. cereus, KRIBB 33587) were purchased from the Korea Collection for Type Cultures (KCTC). Lactobacillus rhamnoses (L. rhamnoses) and Lactobacillus acidophilus (L. acidophilus) were purchased from the Korean Gut Microbiome bank. Staphyloccocus aureus (S. aereus) was purchased from Office Ahn (Gwangju, Republic of Korea). Limosilactobacillus fermentum (L. fermentum) was purchased from the Korean Gut Microbiome Bank (KGMB).

MRS (de Man, Rogosa, Sharp) culture medium in a petri dish was purchased from Kisan Bio, while nutrient medium, microbial culture medium and R2A (Reasoner's 2A agar) medium were purchased from BD DIFCO (Becton, Dickinson and Company, USA). Petri dishes (90×15 mm), snap tubes (15 mL), serological pipettes (5 mL, 10 mL and 25 mL) and spreaders were purchased from SPL Korea, while pipette tips (200 uL) were purchased from Axygen of the USA, and pipettes (30-300 uL) were purchased from Thermo Scientific USA. The vortex mixer was purchased from DLAB MX-S(China), and the Pro KA 33-64AN and Nicrhome Loop 2 mm (KA.11-03A) pipette controllers were purchased from LAB Touch Koryo Ace (Republic of Korea). Microtubes (1.5 mL) were purchased from DeNovo General (Republic of Korea), 1,000 mL solution was purchased from JW-Pharma Saline (Republic of Korea), electric scales (0.001 g) were purchased from Hanyu Electronic Technology E-100 (China)), the magnetic plate stirrer (HP-3000) was purchased from LAB Companion (Republic of Korea), electronic scales (WK-4CII) were purchased from CAS (Republic of Korea), and distilled water was purchased from Joylife (Republic of Korea). The cell culture flasks (25 cm²) were purchased from Falcon (USA), and lactic acid (85-92%) was purchased from Samcheon (Republic of Korea).

The bacteria was cultured at 30° C. in a biochemical incubator (BKSPX-250B III), and observed at magnifications of 40× and 100× using a Leica DM 750 microscope. All solutions and microbes were sterilized before use. All culturing equipment was sterilized using an AC-02 autoclave sterilizer (Jeio Tech, Republic of Korea), and all tests were performed on a clean bench (LT-CB900, Jeio Tech, Republic of Korea) at 23° C.

### Preparation Example 2. Preparation of Kimchi Bacteria Culture Solution

1 colony of L. mesenteroides, L. plantarum, L. brevis, W. koreensis or W. ciberia was added to MRS culture medium (10 mL) in a culture tube (15 mL). The bacteria were stirred using a vortex stirrer, and cultured for 24 hours at 30° C. The culture solution was vortex stirred before using in the next step.

### EXAMPLES

The lower base portion was cut off and the outer leaves were removed from Baechu cabbages (˜7 kg) purchased from a local market. The rinsed Baechu cabbages were cut vertically to around the 1/3 point from the bottom, then split open. The Baechu cabbages were split into 2 to 4 equal parts and placed in a large basin, then sprinkled with coarse sun-dried salt (300 g), angelica (100 g) and Himalayan pink salt (100 g). Luke-warm water (4˜5 cups) was added to brine for 5 to 6 hours, flipping the Baechu cabbages once after 3 hours. The brined Baechu cabbage was rinsed in cold water 2 to 3 times, then drained over a strainer for around 30 minutes.

Peeled white radish (1 kg) was cut into 0.5 cm slices, and spring onion (300 g), celery (200 g) and water parsley (500 g) were cut into pieces not more than 3 cm long. These were tossed lightly with finely minced fermented shrimp (30 g), ground radish, red pepper flakes (200 g), red pepper power paste, salted fermented yellow corvina (100 g), sugar (10 g), corn syrup (20 g), mashed ginger (30 g), mashed garlic (150 g), and mixing paste (Indian rice: Brown rice Glutinous rice powder=1:1:1, stirred into 100 g warm water).

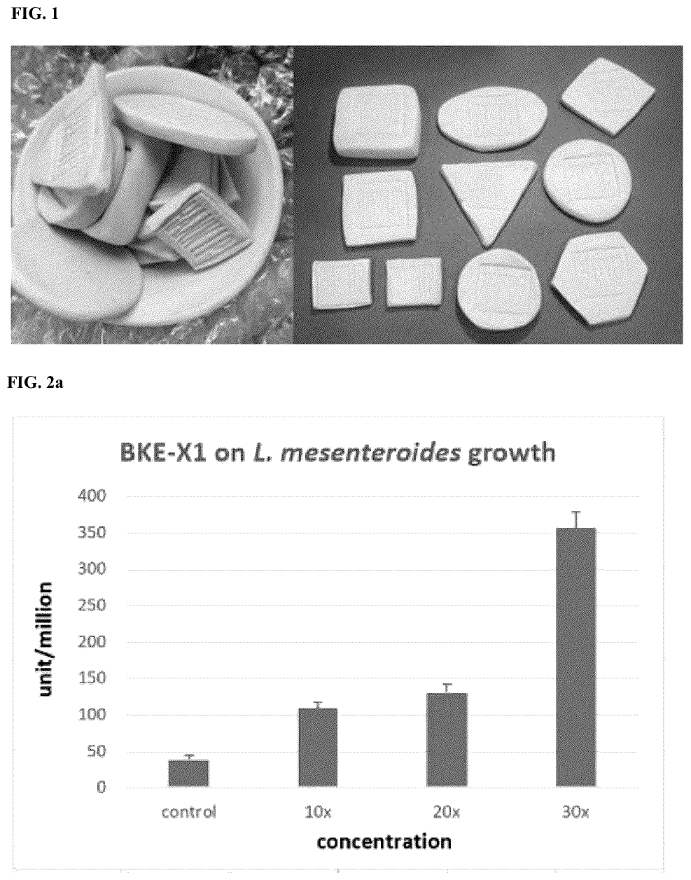

This seasoning paste was spread evenly over the leaves of the brined Baechu cabbages, and after spreading, the Baechu cabbages were wrapped with the outermost leaves and packed into a container. 1 cup (20 g) of salt water was poured over the remaining seasoning paste, and the mixture was poured into the container before closing the lid. The container was left for a day at room temperature to start fermentation, followed by storage in a refrigerator (˜4° C.). During fermentation, germanium-containing ceramic pieces (see FIG. 1) of various sizes and shapes were sterilized and added. Here, the ceramic pieces were prepared by mixing germanium powder with white clay and baking at 800° C., then baking a second time at 1,250° C. (at Doah Studio, Daejeon), and no glaze was applied.

### Example 1. Preparation of BKE-X1

The Baechu cabbage kimchi (200 g) was mashed on day 3 of fermentation, rinsed in water (500 mL) and neutralized to pH 7.0, then filtered (2×) before vacuum concentration (10×) at 85° C.

### Example 2. Preparation of BKE-X2

The Baechu cabbage kimchi (200 g) was mashed on day 15 of fermentation, rinsed in water (500 mL) and neutralized to pH 7.0, then filtered (2×) before vacuum concentration (10×) at 85° C.

### EXPERIMENTAL EXAMPLE

### Experimental Example 1. Determination of Effect of BKE-X1 on Growth of Kimchi Bacteria

Saline solution (9 mL), BKE-X1 (500 mg) and a 0.5 mL aliquot of kimchi bacteria culture solution (select one of L. mesenteroides, L. plantarum, L. brevis, W. ciberia and W. koreensis) was added to MRS culture medium (1 mL) in a culture tube (15 mL), mixed using a vortex stirrer, and cultured for 48 hours at 30° C. Here, as the control, the same ingredients were added to a separate culture medium, excluding BKE-X1, and mixed and cultured using the same method.

An aliquot (0.5 mL) of each sample was collected and serially diluted to 16,000× using saline solution. An aliquot (0.5 mL) was taken from the final diluted solution, plated on an MRS petri dish, and cultured for 48 hours until colonies were visible. The colonies were counted and photographed, then compared side by side.

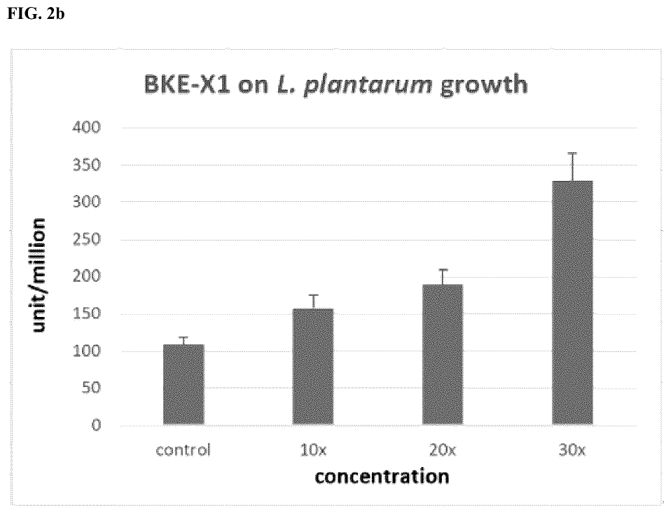

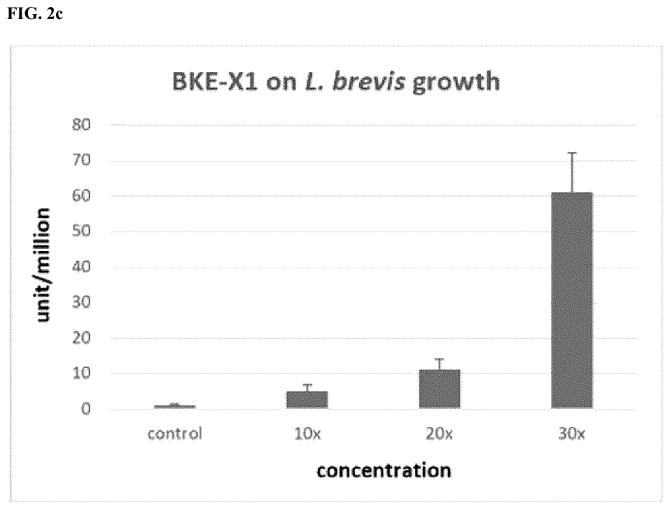

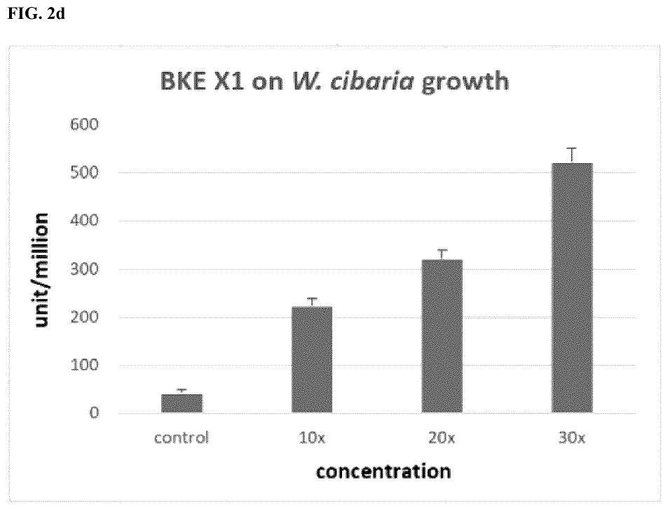

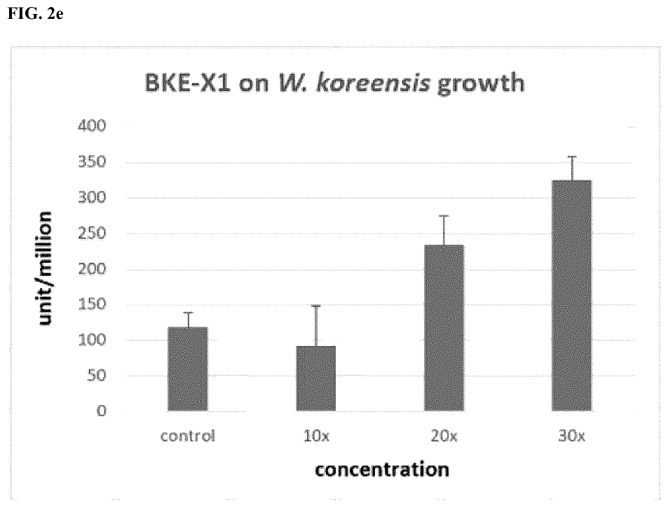

In the results, as shown in FIG. 2a through FIG. 2e, growth of the kimchi bacteria (L. mesenteroides, L. plantarum, L. brevis, W. ciberia and W. koreensis) was effectively promoted as the amount of BKE-X1 increased.

### Experimental Example 2. Determination of Effect of BKE-X2 on Growth of Kimchi Bacteria or Gut Bacteria

Using BKE-X2 instead of BKE-X1 and using kimchi bacteria and/or gut bacteria culture solutions (select one of L. mesenteroides, L. plantarum, L. brevis, W. ciberia, W. koreensis, L. rhamnosus, L. acidophilus and L. fermentum), the method of Experimental Example 1 was repeated.

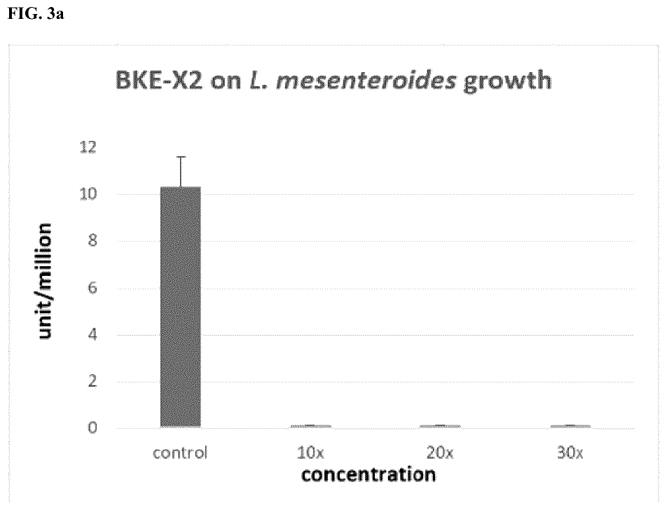

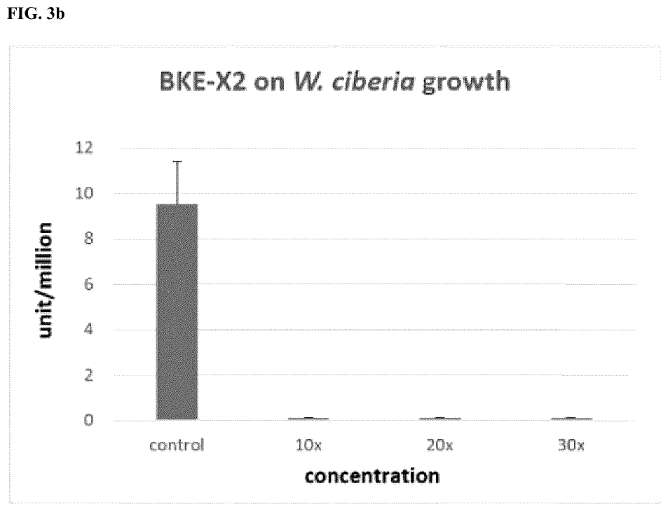

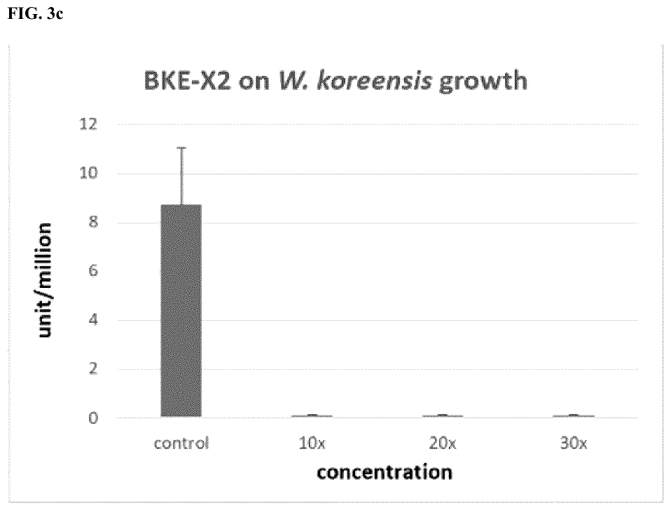

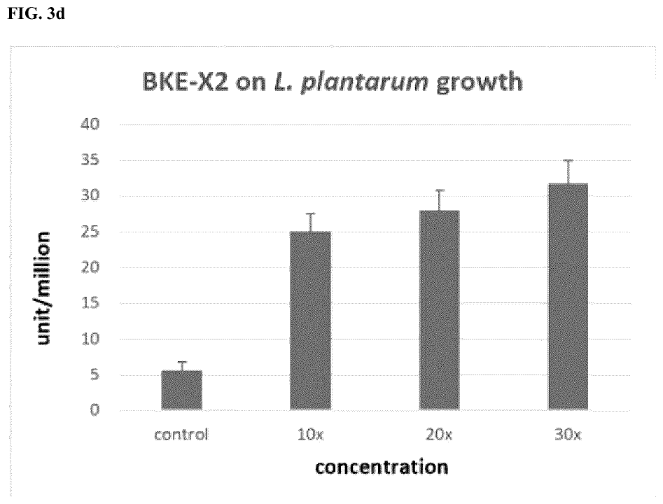

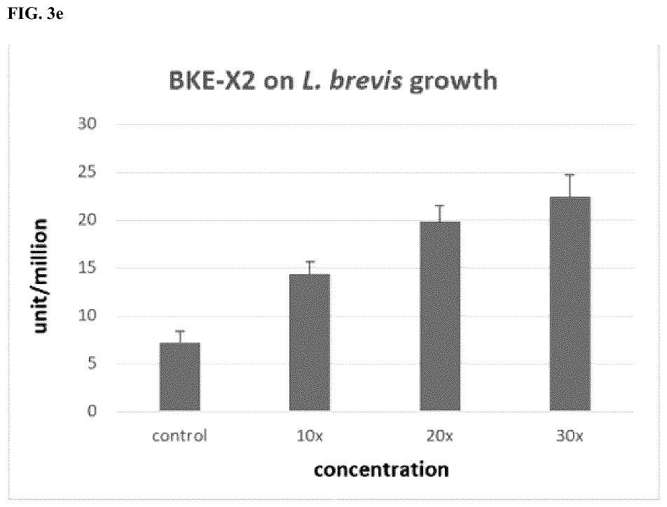

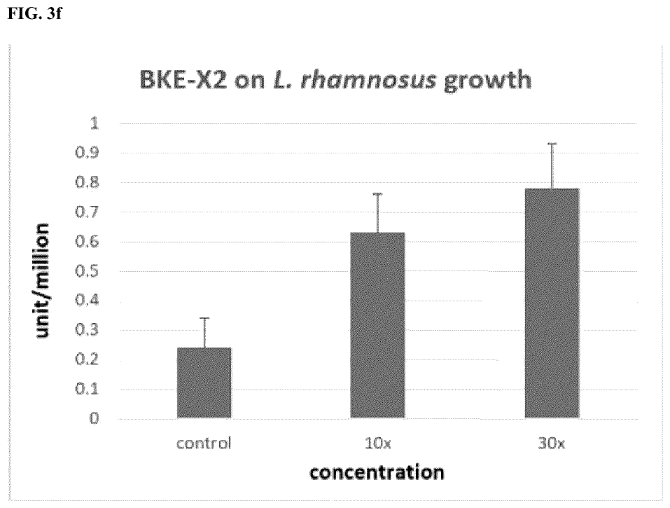

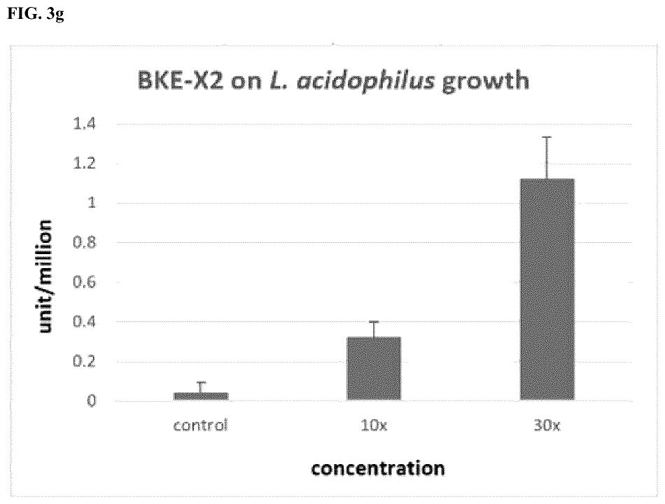

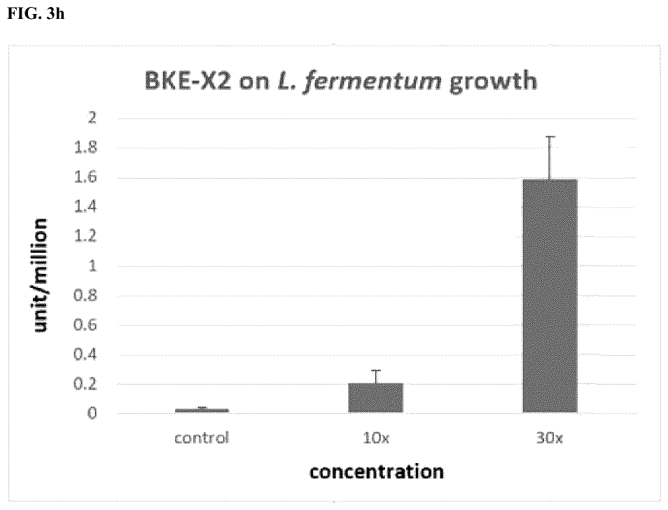

In the results, as shown in FIG. 3, it was found that, as the amount of BKE-X2 increased, whereas the growth of kimchi bacteria (L. mesenteroides, W. koreensis and W. ciberia) was inhibited, growth of gut bacteria (L. plantarum, L. brevis, L. rhamnosus, L. acidophilus and L. fermentum) was effectively promoted.

### Experimental Example 3. Determination of the Impact of Lactic Acid on the Effect of BKE-X1 or BKE-X2 on Growth of Kimchi Bacteria/Gut Bacteria

To MRS culture medium (1 mL) in a culture tube (15 mL), saline (9 mL), BKE-X1 or BKE-X2 (500 mg), and kimchi bacteria culture solution (select one of L. mesenteroides and L. plantarum) (0.5 mL) were added. The mixture was vortex stirred and cultured for 48 hours at 30° C.

To MRS culture medium (1 mL) in another culture tube (15 mL), saline (9 mL), BKE-X1 or BKE-X2 (500 mg), kimchi bacteria culture solution (select one of L. mesenteroides and L. plantarum) (0.5 mL), and lactic acid (to reduce pH to 3 or less) were added. The mixture was vortex stirred and cultured for 48 hours at 30° C.

To MRS culture medium (1 mL) in yet another culture tube (15 mL), saline (9 mL), kimchi bacteria culture solution (select one of L. mesenteroides and L. plantarum) (0.5 mL), and lactic acid (to reduce pH to 3 or less) were added. The mixture was vortex stirred and cultured for 48 hours at 30° C.

As the control, to MRS culture medium (1 mL) in yet another culture tube (15 mL), saline (9 mL) and kimchi bacteria culture solution (select one of L. mesenteroides and L. plantarum) (0.5 mL) were added. The mixture was vortex stirred and cultured for 48 hours at 30° C.

An aliquot (0.5 mL) was collected from each culture medium and serially diluted to 16,000× using saline solution. An aliquot (0.5 mL) was taken from the final diluted solution, plated on an MRS petri dish, and cultured for 48 hours at 30° C. until colonies were visible. The colonies were counted and photographed, then compared side by side.

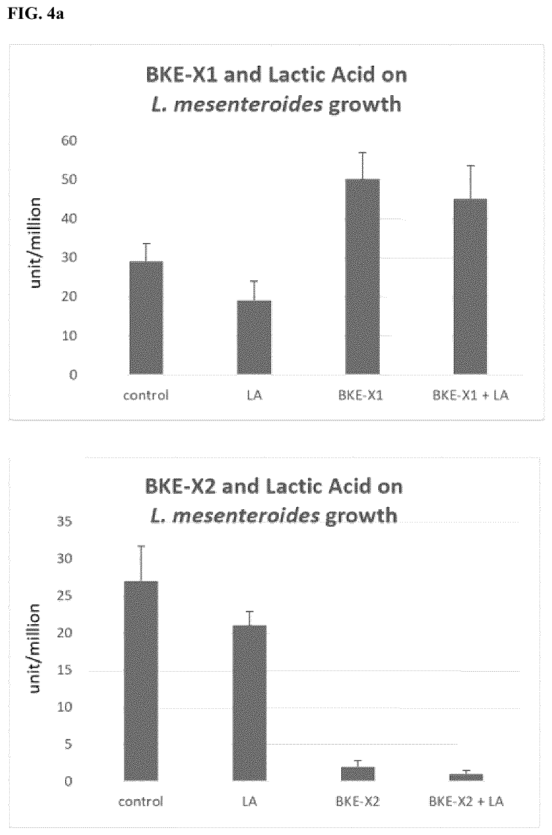

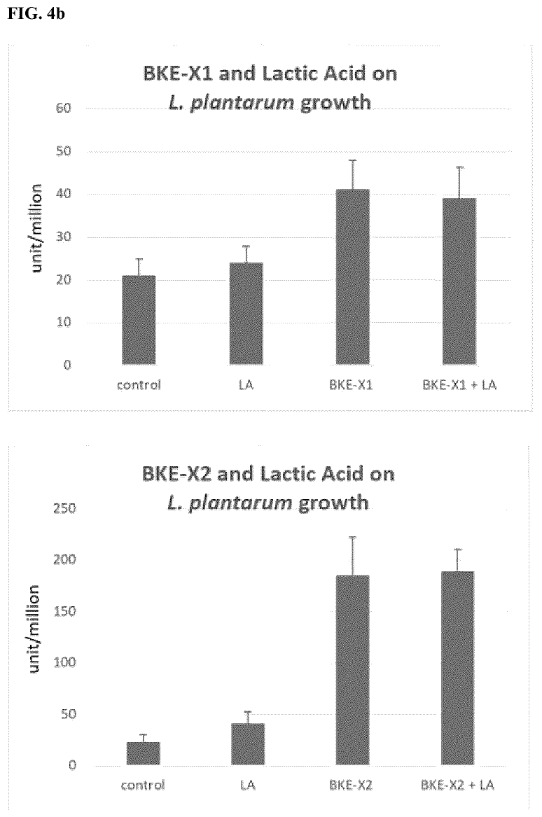

In the results, as shown in FIG. 4, lactic acid was shown to negligibly inhibit the growth of L. mesenteroides while negligibly promoting the growth of L. plantarum. BKE-X1 neutralized the growth inhibition effect of lactic acid on L. mesenteroides, while slightly improving the growth promotion effect of lactic acid on L. plantarum. BKE-X2 greatly enhanced the growth inhibition effect of lactic acid on L. mesenteroides, while also greatly improving the growth promotion effect of lactic acid on L. plantarum.

### Experimental Example 4. Determination of the Effect of BKE-X2 on Growth of Pathogenic Gut Bacteria

4 g of Difco nutrient base was stirred and dissolved for 10 minutes in 500 mL distilled water to prepare a liquid (broth) medium. 4 g of Difco nutrient base and 8 g Difco microbial culture medium were stirred and dissolved for 10 minutes in 500 mL distilled water to prepare an agar culture medium. Both culture mediums were autoclave sterilized for 15 minutes at 121° C.

After autoclave sterilization, the liquid medium as moved to a 15 mL snap tube and the agar base was moved to a cell culture dish at 60° C. Nichrome Loop (11-03A) was used to plate a pathogenic gut bacterium (select one of Escherichia co/i, Proteus mirabilis and Staphylococcus aureus) onto the agar plate, followed by vortex stirring and culturing at 30° C. All equipment and solutions were sterilized before use, and all tests were performed on a clean bench (LT-CB900, Jeio Tech, Republic of Korea) at 23° C. and atmospheric pressure.

From the agar plate, a single colony was collected, then added to nutrient base (1 mL) in a microtube (1.5 mL) using Nichrome Loop (11-03A), vortex stirred, and cultured for 24 hours at 30° C. This culture solution was moved to a 15 mL culture tube, followed by addition of saline (9 mL) and vortex stirring.

50 uL of E. coli culture was moved to a microtube (1.5 mL) (control group), a microtube (1.5 mL) containing BKE-X2 (10 mg) (1% BKE-X2 group), or a microtube (1.5 mL) containing BKE-X2 (30 mg) (3% BKE-X2 group). 100 uL of nutrient solution and 900 uL or saline were added, followed by culturing for 24 hours at 30° C. The E. coli culture solution (50 uL) was plated onto a nutrient agar plate and cultured for 24 hours at 30° C.

After culturing, 50 uL of culture solution was mixed with 10 mL saline in a snap tube and serially diluted twice. The respective serially diluted samples (50 uL) were plated onto a nutrient agar plate, and cultured and kept at 30° C.

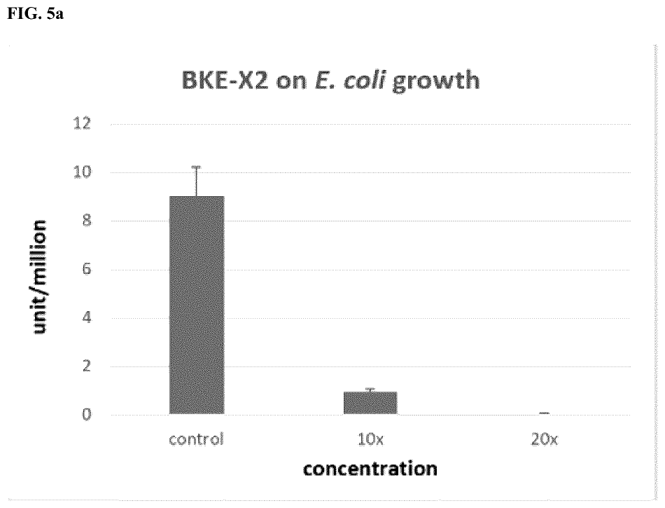

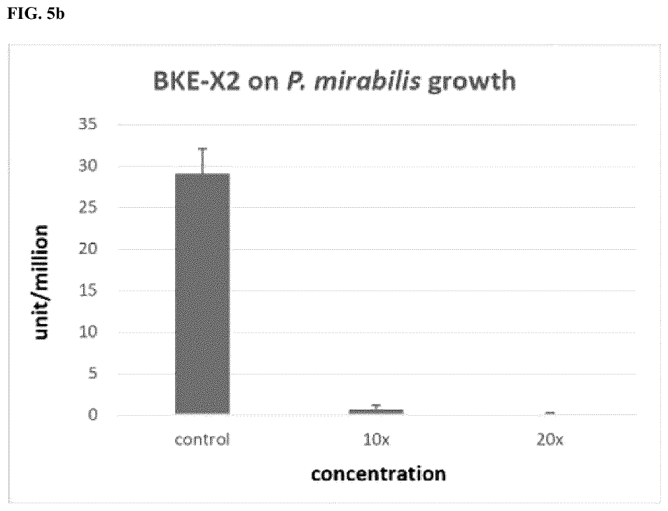

In the results, as shown in FIG. 5a through FIG. 5c, growth of representative pathogenic gut bacteria (E. coli, P. mirabilis and S. aureus) was effectively inhibited at a 10× concentration of BKE-X2.

### Example 5. Growth Inhibition Ring Test for Pathogenic Gut Bacteria

[E. coli, P. mirabilis, S. aureus]

A single colony was collected from agar plates of pathogenic gut bacteria (E. coli, P. mirabilis or S. aureus) cultured using the same method as Example 4, then added to a nutrient base (1 mL) in a microtube (1.5 mL) using a Nichrome Loop (11-03A), vortex stirred, and cultured for 24 hours at 30° C. This culture solution was moved to a 15 mL culture tube, then saline (9 mL) was added and vortex stirred. An aliquot (50 uL) of the culture solution was plated onto a nutrient agar base, followed by addition of BKE-X2 (30˜50 uL each, at 10× and 30× concentrations), and culturing for 18 hours at 30° C.

[Bacillus cereus]

9 g of Difco R2A was stirred and dissolved in 500 mL distilled water for 10 minutes to prepare a growth medium, which was autoclave sterilized for 15 minutes at 121° C. At 60° C., an aliquot (300 uL) of the growth medium was moved to a microtube (1.5 mL), followed by addition of 700 uL saline and vortex stirring. The remaining growth medium was used to prepare an R2A agar plate.

A single colony of B. cereus was collected from a batch growth agar plate, then added to R2A growth medium (1 mL) in a 1.5 mL microtube using a Nichrome Loop (11-03A) and vortex stirred before culturing for 24 hours at 25° C. An aliquot of the culture solution (50 uL) was plated onto an R2A agar plate, followed by addition of BKE-X2 (30˜50 uL each, at 10× and 20× concentrations) and culturing for 16 hours at 30° C. Photographs were taken for growth inhibition ring measurement and visualization.

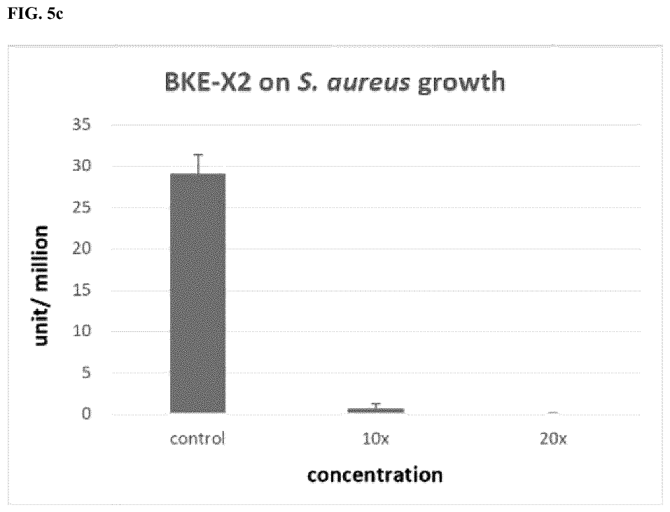

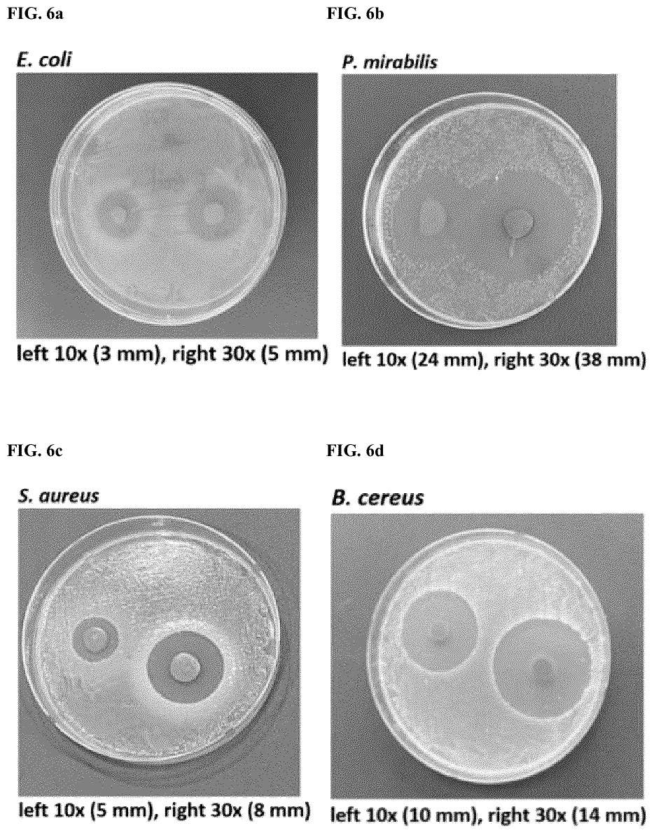

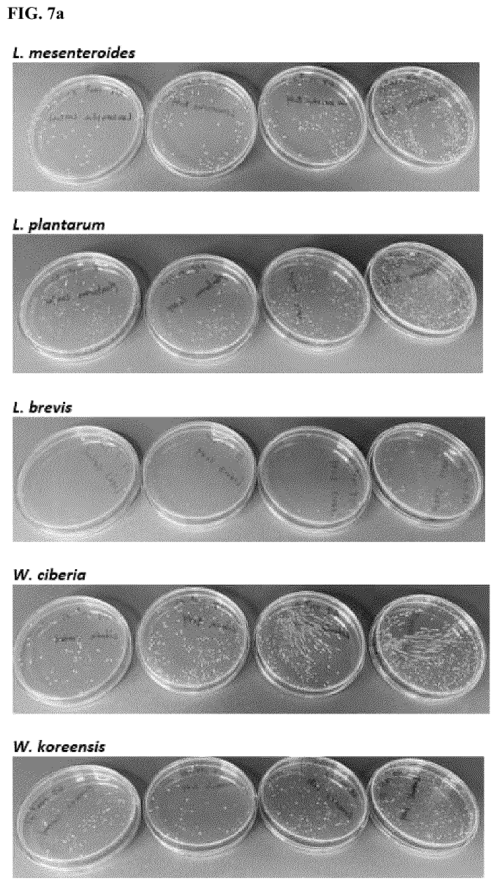

In the results, as shown in FIG. 6, growth of pathogenic gut bacteria was effectively inhibited as the amount of BKE-X2 increased.

### Example 6. Kirby-Bauer Test for BKE-X1 and BKE-X2

An aliquot (50 uL) of kimchi bacteria and/or gut bacteria culture solution (select one of L. mesenteroides, W. ciberia, W. koreensis, L. plantarum, L. brevis, L. rhamnosus, L. acidophilus and L. fermentum) was plated on a nutrient agar plate, treated with varying concentrations (10×, 20× or 30×) of BKE-X1 or BKE-X2, then cultured for 24 hours at 30° C.

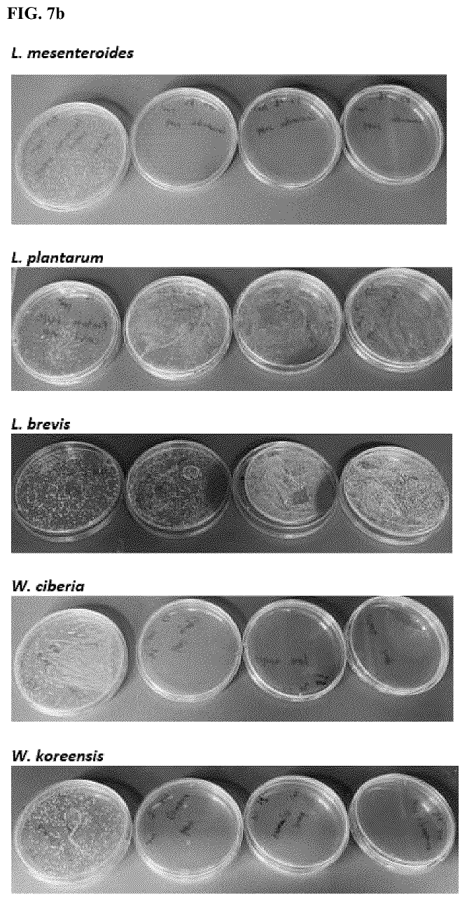

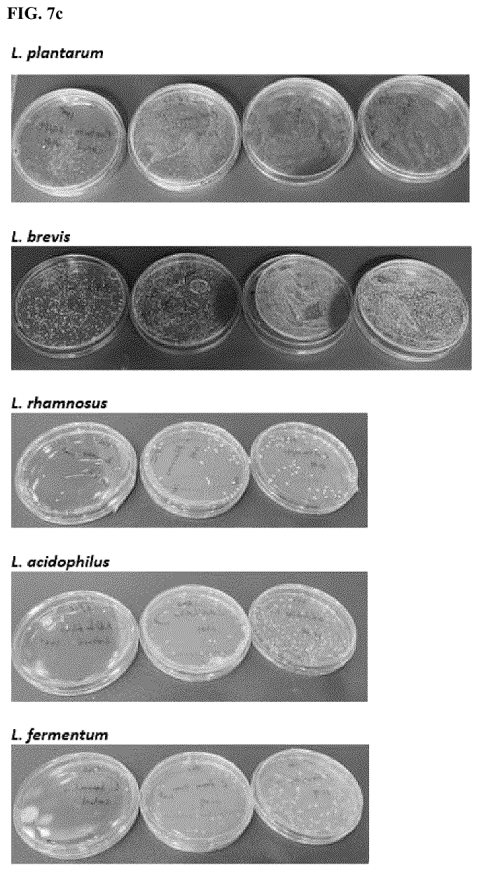

In the results, as shown in FIG. 7a, growth of all kimchi bacteria (L. mesenteroides, L. plantarum, L. brevis, W. koreensis and W. ciberia) was improved as the amount of BKE-X1 increased. Further, as shown in FIG. 7b, as the amount of BKE-X2 increased, the growth of L. plantarum and L. brevis, which are both kimchi bacteria and gut bacteria, was improved, while the growth of the kimchi bacteria L. mesenteroides, W. koreensis and W. ciberia was effectively inhibited. Lastly, as shown in FIG. 7c, as the amount of BKE-X2 increased, growth of all gut bacteria (L. plantarum, L. brevis, L. rhamnosus, L. acidophilus and L. fermentum) was improved.

### CONCLUSION

BKE-X1 according to the present invention was found to be a promoter of growth for L. mesenteroides, Lactobacillus spp. and Weisella spp., while BKE-X2 was found to be a growth inhibitor for L. mesenteroides, Weisella spp. and pathogenic gut bacteria and a growth promoter for L. plantarum, L. brevis, L. rhamnosus, L. acidophilus and L. fermentum.

These results imply that, in the process of fermentation of Baechu cabbage kimchi, a balance between the growth-promoting effects of BKE-X1 and the selective growth-inhibiting effects of BKE-X2 is a key factor determining the bacterial growth patterns in Baechu cabbage kimchi.

1. A method for preparing a concentrated fermented kimchi solution, the method comprising the following steps:
a step of placing kimchi to which a kimchi seasoning paste has been applied into a container with germanium-containing ceramic pieces, then adding salt water and fermenting for 20 to 30 hours at room temperature; and
a step of mashing the kimchi at day 3 or 15 of fermentation, neutralizing, and vacuum concentrating at 80 to 90° C.

2. The method for preparing a concentrated fermented kimchi solution according to claim 1, wherein the germanium-containing ceramic pieces are prepared using a method comprising the following steps:
a step of mixing germanium powder with white clay;
a step of baking at 700 to 900° C., and;
a step of baking a second time at 1,200 to 1,300° C.

3. (canceled)

4. The method for preparing a concentrated fermented kimchi solution according to claim 1, wherein the vacuum concentrating is performed on day 3 of fermentation.

5. The method for preparing a concentrated fermented kimchi solution according to claim 4, wherein the concentrated fermented kimchi solution has a probiotics growth promoting effect.

6. The method for preparing a concentrated fermented kimchi solution according to claim 5, wherein the probiotics are at least one selected from the group comprised of Leuconostoc mesenteroides, Lactobacillus spp. and Weisella spp.

7. The method for preparing a concentrated fermented kimchi solution according to claim 1, wherein the vacuum concentrating is performed on day 15 of fermentation.

8. The method for preparing a concentrated fermented kimchi solution according to claim 7, wherein the concentrated fermented kimchi solution has a probiotics growth promoting effect and/or gut bacteria growth inhibiting effect.

9. The method for preparing a concentrated fermented kimchi solution according to claim 8, wherein the probiotics are at least one selected from the group comprised of Lactobacillus spp. and Limosilactobacillus fermentum.

10. The method for preparing a concentrated fermented kimchi solution according to claim 8, wherein the gut bacteria is at least one selected from the group comprised of Leuconostoc mesenteroides, Weisella spp., and pathogenic gut bacteria.

11. The method for preparing a concentrated fermented kimchi solution according to claim 10, wherein the pathogenic gut bacteria is at least one selected from the group comprised of Escherichia coli, Proteus mirabilis, Staphylococcus aureus and Bacillus cereus.
